(19) United States
(12) Patent Application Publication
(10) Pub. No.: US 2008/0154172 A1
(43) Pub. Date: Jun. 26, 2008

(54) Low Profile Catheters and Methods for Treatment of Chronic Total Occlusions and Other Disorders

(75) Inventor: Kevin Michael Mauch, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/613,764

(22) Filed: Dec. 20, 2006

(51) Int. Cl. A61M 25/09

(52) U.S. Cl. 604/8

(57) ABSTRACT

A catheter having a lumen with a distal end opening and a side opening. A tubular member (e.g., needle or other cannula) is moveable between a retracted position where it is within the catheter lumen proximal to the side opening and an extended position where it extends out of the side opening. When the tubular member is in its retracted position its lumen is substantially coaxial with the catheter lumen such that a guidewire may extend through the lumen of the tubular member and out of the distal end opening of the catheter. The guidewire may then be retracted into the lumen of the tubular member and the tubular member may ten be advanced out of the side opening. Thereafter, the same guidewire (or a different guidewire) may be advanced out of the distal end of the tubular member. Also disclosed are methods for using such catheter to redirect a guidewire or other member and for bypassing an obstruction in a blood vessel such as a chronic total occlusion (CTO) of an artery.

### FIELD OF THE INVENTION

The present invention relates generally to methods and apparatus for medical treatment and more particularly to catheters and related methods for vascular and other transluminal interventional procedures.

### BACKGROUND

### Treatment of Chronic Total Occlusions

Total or near-total occlusions in arteries can prevent all or nearly all of the blood flow through the affected arteries. Chronic total occlusions (CTOs) can occur in coronary as well as peripheral arteries. It has been estimated that approximately 10% of all angioplasty procedures are currently undertaken for CTOs.

In the past, a number of devices have been developed and/or used for the percutaneous interventional treatment of CTOs, such as special guidewires, low-profile balloons, laser emitting wires, atherectomy devices, drills, and drug eluting stents, re-entry catheter, etc. The factor that is most determinative of whether the interventionalist can successfully recannalize a CTO is the interventionalist's ability (or inability) to advance a suitable guidewire from a position within the true lumen of the artery proximal to the CTO, across the CTO lesion (i.e., either through the lesion or around it), and then back into the true lumen of the artery at a location distal to the lesion.

In some instances, such as where the occlusive matter is soft or where the occlusion is less than total, the guidewire can simply be pushed through the occlusive matter itself, thereby allowing the guidewire to remain within the artery lumen. However, in other cases, such as where the artery is totally occluded by hard, calcified atherosclerotic plaque, the guidewire may tend to deviate to one side and penetrate through the intima of the artery, thereby creating a neo-lumen called a “subintimal space” (e.g., a penetration tract formed within the wall of the artery between the intima and adventitia). In these cases, the distal end of the guidewire may be advanced to a position distal to the lesion but remains entrapped within the subintimal space. In such instances, it is then necessary to divert or steer the guidewire from the subintimal space back into the true lumen of the artery at a location distal to the CTO lesion.

### Catheters Useable to Facilitate Reentry into True Lumen

A number of catheters based devices have been heretofore useable to redirect subintimally entrapped guidewires back into the true lumen of the artery. Included among these are the devices described in U.S. Pat. Nos. 5,830,222 (Makower), 6,068,638 (Makower), 6,159,225 (Makower), 6,190,353 (Makower, et al.), 6,283,951 (Flaherty, et al.), 6,375,615 (Flaherty, et al.), 6,508,824 (Flaherty, et al.), 6,544,230 (Flaherty, et al.), 6,655,386 (Makower et al.), 6,579,311 (Makower), 6,602,241 (Makower, et al.), 6,655,386 (Makower, et al.), 6,660,024 (Flaherty, et al.), 6,685,648 (Flaherty, et al.), 6,709,444 (Makower), 6,726,677 (Flaherty, et al.) and 6,746,464 (Makower) describe a variety of catheters having laterally deployable cannulae (e.g., hollow needles). These catheters are advanced into the subintimal space, over the subintimally entrapped guidewire. Thereafter, the laterally deployable cannula is advanced from the catheter into the true lumen of the blood vessel, downstream of the CTO. A second guidewire is then passed through that laterally deployed cannula and is advanced into the true lumen of the artery. The laterally deployed cannula is then retracted into the catheter and the catheter is removed, along with the original guidewire, leaving just the second guidewire in place. This second guidewire is then useable to facilitate enlargement (e.g., balloon dilation, atherectomy, etc.) and/or stenting of the subintimal space, thereby creating a sub-intimal bypass conduit around the CTO. These types of catheter devices are also useable in many types of interventions, including the delivery of substances (e.g., drugs, biologics, cells, genes, contrast media or other diagnostic or therapeutic substances), articles or devices to target locations within the body, passage of guidewires and/or catheters for accessing target locations, bypassing of obstructions, re-entry into a true lumen of a blood vessel from a subintimal space, etc. Commercially available catheters of this type have been used successfully in the treatment of CTOs in relatively large vessels, such as the femoral artery, popliteal artery, etc. However, their use in coronary vessels and other small vessels has been limited to date due to their relatively large diameter.

There exists a need in the art for the development of new small diameter catheters that have laterally deployable members (e.g., cannulae, needles, probes, wires, etc.) which may be used to redirecting subintimally entrapped guidewires and/or for other purposes such as delivery of substances, articles or devices to specific target locations within the body.

### SUMMARY OF THE INVENTION

In accordance with the present invention there is provided a catheter device comprising an elongate catheter body having a lumen with a distal end opening and a side opening and a tubular member (e.g., a needle or other cannula) that has a lumen and an open distal end. The tubular member is moveable back and forth between (a) a retracted position where the open distal end of the tubular member is within the lumen of the catheter body and (b) an extended position wherein a distal portion of the tubular member is advanced out of the side opening of the catheter body. In some embodiments, the catheter may have only a single lumen and the catheter body may have an outer diameter of less than 0.050 inch.

Further in accordance with the invention, there is disclosed a method for redirecting a guidewire from a first position within the body of a human or animal subject to a second position within the body of the subject. Such method is performed using a catheter of the type summarized in the immediately preceding paragraph. The proximal end of the guidewire is inserted into the distal end opening of the catheter body and the catheter is advanced over the guidewire while the tubular member is in its retracted position, thereby causing the proximal end of the guidewire to pass through the lumen of the tubular member. Thereafter, the guidewire is retracted to a position where the distal end of the guidewire is proximal to the distal end of the tubular member (or in some cases it may be removed completely). Then, the tubular member to its extended position such that the open distal end of the tubular member is at or in substantial alignment with the second location. Thereafter, the guidewire (or a different guidewire) is advanced out of the open distal end of tubular member and into the second location.

Further in accordance with the invention, there is provided a method for bypassing an obstruction in a blood vessel (e.g., a chronic total occlusion or other full or partial obstruction). In this method, a guidewire is advanced into the blood vessel such that the distal end of the guidewire becomes positioned within a subintimal space. Thereafter, a catheter device of the type referred to in the two immediately preceding paragraphs is advanced over the guidewire while its hollow needle is in the retracted position. This results in passage of a proximal portion of the guidewire into the lumen of the hollow needle. With the catheter positioned in the subintimal space, the guidewire is then retracted in the proximal direction such that the distal end of the guidewire is within the lumen of the needle. (In some cases, the guidewire may be fully retracted, removed, and another guidewire may be inserted into the lumen of the hollow needle. However, such guidewire exchange is optional and not required, as this catheter is fully functional with a single guidewire, as described herein.) The hollow needle is then advanced to its extended position whereby the open distal end of the needle is within the true lumen of the blood vessel downstream of the obstruction. The guidewire is then advanced from the lumen of the hollow needle into the true lumen of the blood vessel downstream of the obstruction. Because the catheters of the present invention may be of relatively small diameter, some embodiments of such catheters may be used by this method for treatment of coronary CTOs as well as CTO in larger peripheral arteries.

Further aspects, details and embodiments of the present invention will be understood by those of skill in the art upon reading the following detailed description of the invention and the accompanying drawings.

### BRIEF DESCRIPTION OF THE DRAWINGS

FIG. 1 is a side view of one embodiment of a catheter device of the present invention.

FIG. 2 is a partial cut away view of the distal end of the catheter device of FIG. 1.

FIG. 2A is a cross-sectional view through line 2A-2A of FIG. 2.

FIG. 3 is a partial cut away view of the distal end of the catheter device of FIG. 1 incorporating optional deflector apparatus.

FIG. 4 is an anatomical diagram showing the histological layers of an artery.

FIGS. 4A-4G show steps in a method for using the catheter device of FIG. 1 to perform a transluminal, catheter-based bypass of a CTO in an artery.

### DETAILED DESCRIPTION

In the following detailed description, the accompanying drawings are intended to describe some, but not necessarily all, examples or embodiments of the invention. The contents of this detailed description and accompanying drawings do not limit the scope of the invention in any way.

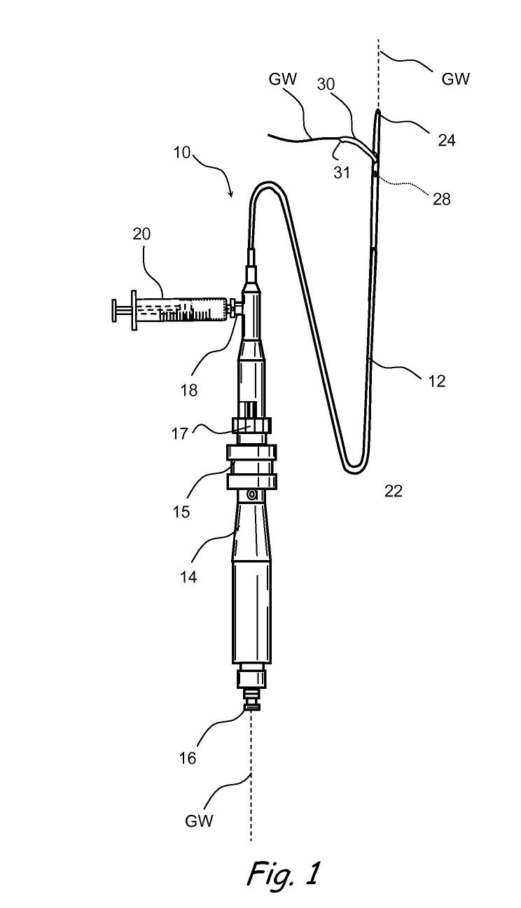

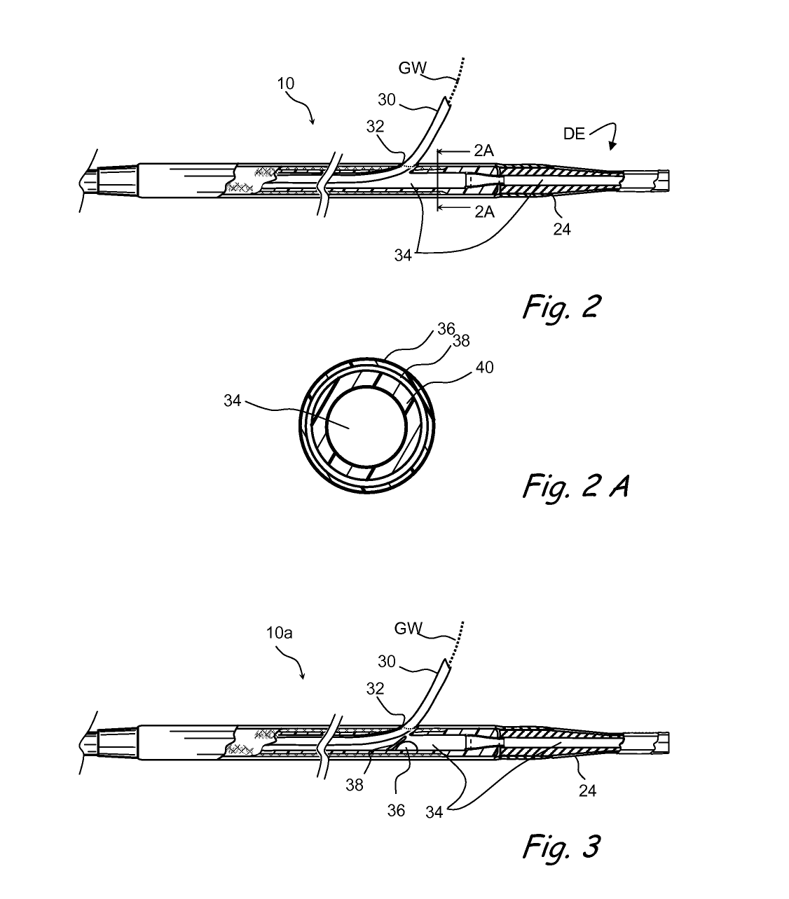

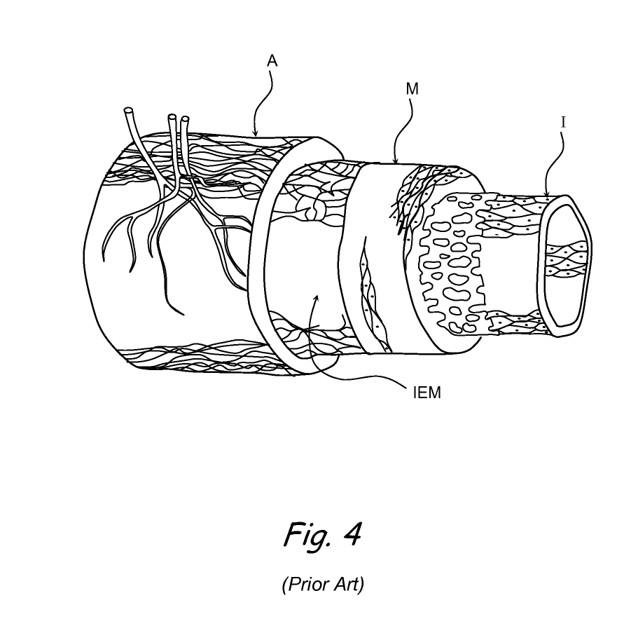

FIGS. 1-2A show an example of a catheter device 10 of the present invention. This catheter device 10 comprises an elongate catheter body 12 (e.g., a catheter shaft) having an atraumatic distal tip 24 on its distal end DE and a handpiece 14 on its proximal end. As seen in the more detailed views of FIGS. 2 and 2A, some embodiments of the catheter body 12 may comprises a core member 40, a braid layer 38 surrounding the core member 40 and an outer layer 36 surrounding the braid layer 38. Possible materials for the components are as follows: core member 40 (HDPE, LLDPE, LDPE, polyimide (PI), Pebax, Nylon, PEEK), braid layer 38 (stainless steel, nitinol, polymeric and/or glass reinforced fibers (e.g. Kevlar), outer layer 36 (HDPE, LLDPE, LDPE, polymide (PI), Pebax, Nylon, PEEK. In the configuration where the needle/hypotube serves as the guidewire the possible dimensions of the resulting components may be as follows: core member 40 (ID=0.020″, OD=0.026″), braid layer 38 (thickness<0.005″), outer layer 36 (thickness<0.005″). Resulting overall shaft profile OD<0.046″). A through lumen 34 extends from a port 16 on the proximal end of the handpiece 14, through the handpiece 14, through the catheter body 12, and through the distal tip member 24, terminating in a distal end opening. Additionally, a side opening 32 is formed in the catheter body 12 in communication with lumen 34.

A tubular member 30 (e.g., a hollow needle or other cannula) having an open distal end 31 is moveable back and forth between a) a retracted position where the open distal 31 end of the tubular member 30 is within the lumen 34 of the catheter body 12 and b) an extended position wherein a distal portion of the tubular member 30 is advanced out of the side opening 32 of the catheter body 12 as seen in the showing of FIG. 2. In some embodiments, this tubular member may be formed of elastic or supereleastic material (e.g., nickel-titanium alloy) and a distal portion of this tubular member 30 may be biased to a curved configuration, such as that seen in FIG. 2. In the example shown in the drawings, a knob 15 on handpiece 14 may be moved (continuously or incrementally) in the distal direction to cause advancement of the tubular member 30 to its extended position and retracted (continuously or incrementally) in the proximal direction to cause retraction of the tubular member 30 to its retracted position. Optionally, a fixed or adjustable stop apparatus 17 may be included on handpiece 14 or elsewhere to limit the extent to which the tubular member 30 may be advanced (e.g., thereby limiting the length of the portion of the tubular member 30 that extends out of side opening 32 when the tubular member 30 is in its fully extended position).

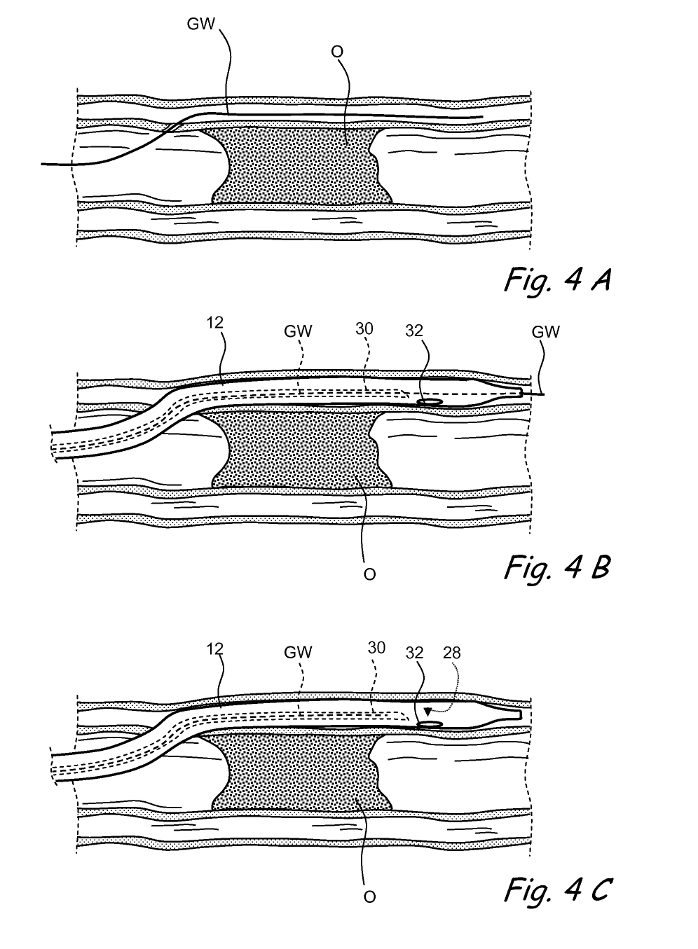

In some embodiments, the tubular member 30 may be biased to a curved configuration which causes its distal end 31 to pass out of the side opening 32 as the tubular member 30 is advanced in the distal direction through lumen 34, as seen in FIG. 2. In other embodiments, such as the alternative embodiment shown in FIG. 3, a deflector 38 may be deployed to deflect the distal end 31 of the tubular member 30 out of side opening 32, when desired. In the particular example of FIG. 2B, the deflector 38 becomes raised when a balloon 36 is inflated by the operator. When the deflector 38 is raised, the distal end 31 of the advancing tubular member 30 will strike the surface of the deflector and will be thereby deflected out of side opening 32, as seen in FIG. 2B.

Optionally, the catheter device 10 may incorporate an orientation indicating element 28 which indicates the radial direction in which, or the trajectory on which, the tubular member 30 will advance from the catheter body12. In some embodiments, this orientation indicating element 28 may comprise a marker that is imageable by an imaging apparatus that is located on/in the catheter body 12 or elsewhere (e.g., a fluoroscope or other extracorporeal imaging device). In some embodiments, this orientation indicating element 28 may comprise an imaging apparatus that images the target location to which it is desired to advance the tubular member 30 (e.g., the true lumen of an artery) along with an electronic or physical indicator of the direction in which, or the trajectory on which, the tubular member 30 will advance from the catheter body12. Thus, this orientation indicating element 28 provides information which the operator may use to make any necessary adjustments in the position and rotational orientation of the catheter body 12 in situ ensure (or at least increases the probability) that the tubular member 30 will subsequently advance to the intended target location and not some other location. Examples of the various types of orientation elements 28 that may be used include but are not limited to those described in U.S. Pat. Nos. 5,830,222 (Makower), 6,068,638 (Makower), 6,159,225 (Makower), 6,190,353 (Makower, et al.), 6,283,951 (Flaherty, et al.), 6,375,615 (Flaherty, et al.), 6,508,824 (Flaherty, et al.), 6,544,230 (Flaherty, et al.), 6,655,386 (Makower et al.), 6,579,311 (Makower), 6,602,241 (Makower, et al.), 6,655,386 (Makower, et al.), 6,660,024 (Flaherty, et al.), 6,685,648 (Flaherty, et al.), 6,709,444 (Makower), 6,726,677 (Flaherty, et al.) and 6,746,464 (Makower), which are incorporated herein by reference.

In operation, while the tubular member 30 is in its retracted position, it will be positioned substantially coaxially within lumen 34 of the catheter body 12 such that a guide wire GW may extend from proximal port 16, through handpiece 14, though lumen 34 and out of the open distal end of tip member 24. Thereafter the guidewire GW may be retracted in the proximal direction until the distal end of the guidewire GW is proximal to the distal end 31 of the tubular member 30. Thereafter, the tubular member 30 may be moved from its retracted position to its extended position, such that a distal portion of the tubular member 30 extends out of side opening 32. The guidewire GW may then be advanced in the distal direction and out of the open distal end 31 of the tubular member.

Optionally, an infusion/aspiration port 18 may be in communication with the lumen 34 of the catheter body 12 or the lumen of the tubular member 30 to permit infusion or aspiration of matter through the lumen 34 of the catheter body 12 or the lumen of the tubular member 30. A Luer fitting or other suitable connector may be provided to facilitate connection of a syringe 20, solution administration tube or other infusion or aspiration device to port 18. Also optionally, a valve (e.g., a Tuohy-Borst valve), cap or other closure apparatus (not shown) may be associated with port 16 to deter backflow of fluids out of port 16 when fluids are being infused through port 18. Port 16 also provides access to the lumen of tubular member 30. This allows for the insertion of guidewires, mandrels, and fluids through tubular member 30.

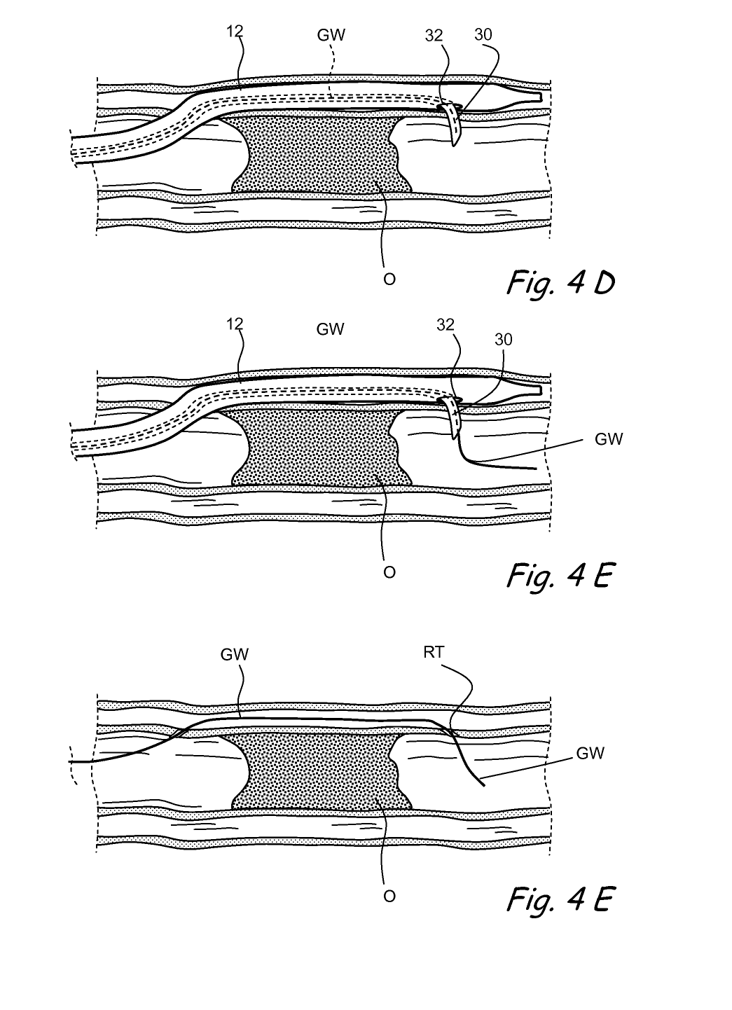

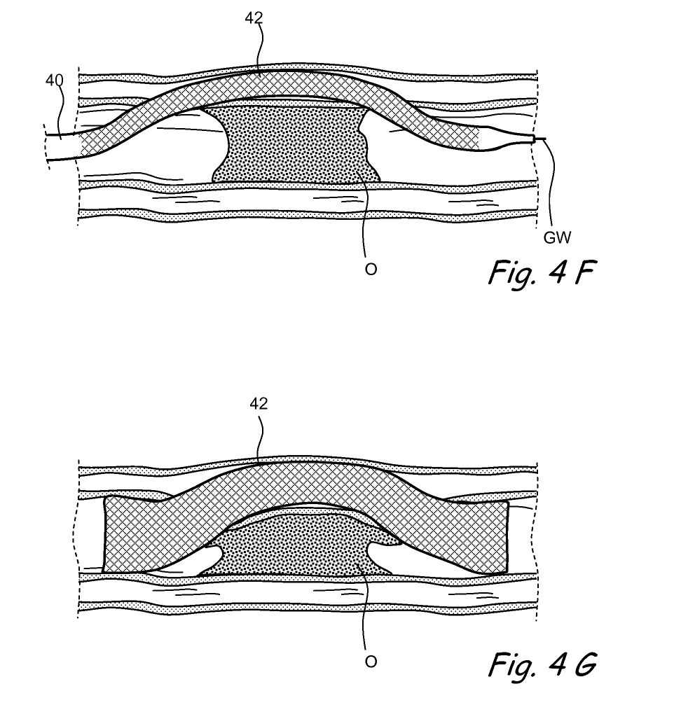

FIGS. 4-4G show an example of a procedure in which the above-described catheter device 10 is used to treat a CTO of an artery.

As specifically shown in FIG. 4, the wall of an artery typically consists of three layers, the tunica intima (“intima”), tunica media M (“media”) and the tunica adventitia A (adventitia). In some arteries an internal elastic membrane IEM is disposed between the media M and adventitia A.

Initially, as shown in FIG. 4A and in accordance with techniques well known in the field of interventional cardiology and/or interventional radiology, a guidewire 26 is advanced into a subintimal space adjacent to an obstruction ◯ such that the distal end of the guidewire 26 is within the subintimal space, distal to the obstruction ◯.

Thereafter, As seen in FIG. 4B, the catheter 10 of the present invention is advanced over the guidewire GW while the tubular member 30 is in its retracted position within lumen 34 such that the distal end 31 of the tubular member 30 is proximal to side opening 32. The catheter body 12 is positioned such that the side outlet opening 32 is distal to the obstruction ◯. Before or after such retraction of the guidewire GW, if the optional orientation indicating element 28 is present, it may be used by the operator to make any necessary adjustment of the rotational orientation of the catheter body 12 within the subintimal space to ensure, or to at least increase the probability that, subsequent advancement of the tubular member 30 to its extended position will cause the distal end 31 of the tubular member 30 to enter the true lumen TL of the artery, distal to the obstruction ◯.

Thereafter, as seen in FIG. 4C, the tubular member 30 is then advanced out of side opening 32, through adjacent tissue, and into the true lumen TL of the artery, distal to the obstruction ◯.

Thereafter, as shown in FIG. 4D, guidewire GW is advanced through the lumen of tubular member 30, out of the distal end 31 of the tubular member 30 and into the true lumen TL of the artery. Optionally, in some cases, it may be desirable to remove and exchange the guidewire GW before it is advanced through tubular member 30 and into the true lumen TL of the artery. For example, in some applications a specialized guidewire may be forced into the artery wall to create the subintimal space and to guide the initial advancement of the catheter body 12 into the subintimal space. However, it may be desire to use a different guidewire (e.g., a more flexible guidewire) for reentry into the true lumen. In such cases, the first guidewire may be fully removed in FIG. 4C and a second guidewire may then be advanced through the lumen of the tubular member 30 in FIG. 4D.

Subsequently, as seen in FIG. 4E, the tubular member 30 is withdrawn to its retracted position and the catheter 10 is removed, leaving the guidewire GW in place such that it extends through the true lumen TL of the artery proximal to (i.e., upstream of) the obstruction ◯, through the subintimal space, through the reentry tract RT created by advancement of the tubular member 30 in FIG. 4D and back into the true lumen TL of the artery distal to (i.e., downstream of) the obstruction ◯.

One or more tract modifying devices (e.g., balloon catheters, atherectomy catheters, stent delivery catheters, laser catheters, etc.) may then be advanced over the guidewire and used to enlarge (e.g., dilate, debulk, bore, stent, etc.) the subintimal space. For example, FIG. 4F shows a balloon catheter 40 having a stent 42 mounted thereon being advanced over the guidewire GW to a position where one end of the stent 42 is in the true lumen TL proximal to the obstruction ◯ and the other end of the stent 42 is in the true lumen TL distal to the obstruction ◯. The balloon of the balloon catheter 40 is then used to dilate the subintimal space and to expand the stent 42. Thereafter, the balloon is deflated and the balloon catheter 40 is removed, leaving the stent 42 in an expanded configuration and creating a stented, subintimal bloodflow channel around the obstruction ◯ as seen in FIG. 4G.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. A catheter device comprising:
an elongate catheter body having a lumen with a distal end opening and a side opening;
a tubular member having a lumen and an open distal end, said tubular member being moveable back and forth between a) a retracted position where the open distal end of the tubular member is within the lumen of the catheter body and b) an extended position wherein a distal portion of the tubular member is advanced out of the side opening of the catheter body.

2. A device according to claim 1 wherein the tubular member comprises a cannula.

3. A device according to claim 1 wherein the tubular member comprises a hollow needle.

4. A device according to claim 1 wherein the tubular member is biased to a curved configuration and such bias causes the distal end of the tubular member to pass out of the side opening as the tubular member is advanced in the distal direction through the lumen of the catheter body.

5. A device according to claim 1 further comprising a deflector for deflecting the tubular member out of the side opening as the tubular member is advanced in the distal direction through the lumen of the catheter body.

6. A device according to claim 1 or 4 wherein the tubular member is formed substantially of material which is superelastic at normal body temperature.

7. A device according to claim 6 wherein said material comprises a nickel-titanium alloy.

8. A device according to claim 1 further comprising apparatus for limiting advancement of the tubular member out of the side opening.

9. A device according to claim 1 wherein said catheter body lumen is the only lumen formed in the catheter body.

10. A device according to claim 1 wherein the catheter body has an outer diameter of less than 0.050 inch.

11. A device according to claim 1 further comprising at least one orientation apparatus useable to provide an indication of the direction or trajectory on which the tubular member will advance from the catheter body.

12. A system comprising a device according to claim 1 further in combination with a guidewire that is sized to pass through the lumen of the tubular member.

13. A method for bypassing an obstruction in a blood vessel that has a true lumen and a vessel wall, and wherein the vessel wall has an intima, said method comprising the steps of:
(A) providing a guidewire that has a proximal end and a distal end;
(B) positioning the guidewire such that its distal end is within a subintimal space and its proximal end is outside of the subject's body;
(C) providing a catheter device that comprises
i) an elongate catheter body having a lumen with a distal end opening and a side opening; and
ii) a tubular member having a lumen and an open distal end, said tubular member being moveable back and forth between a) a retracted position where the open distal end of the tubular member is within the lumen of the catheter body and b) an extended position wherein a distal portion of the tubular member is advanced out of the side opening of the catheter body;
(D) inserting the proximal end of the guidewire into the distal end opening of the catheter body and advancing the catheter over the guidewire while the tubular member is in its retracted position, thereby causing the proximal end of the guidewire to pass through the lumen of the tubular member;
(E) retracting the guidewire to a position where the distal end of the guidewire is proximal to the distal end of the tubular member;
(F) moving the tubular member to its extended position such that the open distal end of the tubular member enters the true lumen of the blood vessel distal to the obstruction;
(G) advancing the guidewire out of the open distal end of tubular member and into the true lumen of the blood vessel distal to the obstruction;
(H) retracting the tubular member to its retracted position;
(I) removing the catheter device, leaving the guidewire in place; and
(J) advancing at least one working device over the guidewire and using said at least one working device to modify the subintimal space to create a patent bypass channel through which blood may flow around the obstruction.

14. A method according to claim 13 wherein the obstruction is a chronic total occlusion of at least one artery.

15. A method according to claim 14 wherein the at least one artery is at least one peripheral artery.

16. A method according to claim 14 wherein the at least one peripheral artery is the iliac artery, the femoral artery, the popliteal artery or a segment of vasculature that includes portion of the femoral and popliteal arteries.

17. A method according to claim 14 wherein the at least one artery is a coronary artery.

18. A method according to claim 13 wherein the catheter device further comprises at least one orientation apparatus that provides an indication of the direction or trajectory on which the tubular member will advance or extend from the catheter body and wherein, prior to performance of Step F, the method further comprises the step of:
using the orientation apparatus to guide any necessary adjustment of the rotational orientation of the catheter body to thereby increase the likelihood that the tubular member will subsequently enter the true lumen of the blood vessel rather than some other location.

19. A method according to claim 13 wherein:
Step E comprises removing the guidewire and inserting a replacement guidewire having a distal end into the lumen of the tubular member such that the distal end of the replacement guidewire is proximal to the open distal end of the tubular member; and
the guidewire referred to in subsequent Steps G, I and J is the replacement guidewire.

20. A method according to claim 13 wherein Step J comprises advancing a balloon over the guidewire and using the balloon to dilate the subintimal space.

21. A method according to claim 13 wherein Step J comprises advancing an atherectomy device over the guidewire and using the atherectomy device to enlarge the subintimal space.

22. A method according to claim 13 wherein Step J comprises advancing a stent over the second guidewire and causing the stent to expand within the subintimal space.

23. A method according to claim 13 wherein the catheter body has a single lumen and an outer diameter of less than 0.050 inch.

24. A method according to claim 23 wherein the blood vessel is a coronary artery.

25. A method for redirecting a guidewire from a first position within the body of a human or animal subject to a second position within the body of the subject, said method comprising the steps of:
(A) providing a catheter device that comprises
iii) an elongate catheter body having a lumen with a distal end opening and a side opening; and
iv) a tubular member having a lumen and an open distal end, said tubular member being moveable back and forth between a) a retracted position where the open distal end of the tubular member is within the lumen of the catheter body and b) an extended position wherein a distal portion of the tubular member is advanced out of the side opening of the catheter body;
(B) inserting the proximal end of the guidewire into the distal end opening of the catheter body and advancing the catheter over the guidewire while the tubular member is in its retracted position, thereby causing the proximal end of the guidewire to pass through the lumen of the tubular member;
(C) retracting the guidewire to a position where the distal end of the guidewire is proximal to the distal end of the tubular member;
(D) moving the tubular member to its extended position such that the open distal end of the tubular member is at or in substantial alignment with the second location;
(E) advancing the guidewire out of the open distal end of tubular member and into the second location.

26. A method according to claim 23 further comprising the steps of:
(F) retracting the tubular member to its retracted position; and
(G) removing the catheter leaving the guidewire in place with the distal end of the guidewire at the second location.

27. A method according to claim 24 further comprising the step of:
(H) advancing a working device over the guidewire to said second location.

28. A method according to claim 23 wherein the catheter device further comprises at least one orientation apparatus that is useable to provide an indication of the direction or trajectory on which the tubular member will advance or extend from the catheter body and wherein, prior to performance of Step E, the method further comprises the step of:
using the orientation apparatus to make any necessary adjustment of the rotational orientation of the catheter body to thereby increase the likelihood that the tubular member will subsequently enter the true lumen of the blood vessel rather than some other location.
